SEQUENCE LISTING (4 sequences)

SEQ ID NO: 1 - DNA, 25 bp, SARS-COV-2
  source 1..25: mol_type = genomic DNA
agaagattggttagatgatgatagt

SEQ ID NO: 2 - DNA, 25 bp, SARS-COV-2
  source 1..25: mol_type = genomic DNA
ttccatctctaattgaggttgaacc

SEQ ID NO: 3 - DNA, 24 bp, SARS-COV-2
  source 1..24: mol_type = genomic DNA
tcctcactgccgtcttgttgacca

SEQ ID NO: 4 - protein, 12 aa, synthetic construct
  REGION 1..12: note = Synthetic
ESATLQSGLAKS

(19) United States
(12) Patent Application Publication
(10) Pub. No.: US 2025/0082667 A1
(43) Pub. Date: Mar. 13, 2025

(54) THERAPEUTIC AGENTS FOR TREATMENT OF CORONAVIRUS INFECTION

(71) Applicant: Xueyun Gao, Beijing (CN)

(72) Inventor: Xueyun Gao, Beijing (CN)

(21) Appl. No.: 18/244,800

(22) Filed: Sep. 11, 2023

Related U.S. Application Data
(63) Continuation of application No. 17/181,597, filed on Feb. 22, 2021, now abandoned.
(60) Provisional application No. 63/030,526, filed on May 27, 2020.
(60) Provisional application No. 63/045,128, filed on Jun. 28, 2020.

(51) Int. Cl. A61K 31/7135; A61K 9/00; A61K 47/64; A61P 31/14; C07K 5/083

(52) U.S. Cl. CPC A61K 31/7135; A61K 9/0019; A61K 47/64; A61P 31/14; C07K 5/0806

(57) ABSTRACT

A therapeutic method for treatment of coronavirus infections. The therapeutic method includes using a composition containing a gold compound effective for Rheumatoid Arthritis treatment that both generates gold-S bonds at the active pockets of the main protease of the virus and suppresses the virus induced inflammations in the body.

### CROSS-REFERENCE

This is a divisional application to U.S. patent application Ser. No. 17/181,597 filed on Feb. 22, 2021, which claims priority of the U.S. Provisional Patent Application No. 63/030,526, filed on May 27, 2020, entitled “Molecules inhibit the enzyme activity of 3-chymotrypsin-like cysteine protease of SARS-CoV-2 virus,” the entirety of which is hereby incorporated by reference for all purposes. Priority is also claimed from the U.S. Provisional Patent Application No. 63/045,128, filed on Jun. 28, 2020, entitled “Au ions bind the Cys145 and Cys156 residue of the 3-chymotrypsin-like cysteine protease of SARS-CoV-2 and inhibit it activity,” the entirety of which is hereby incorporated by reference for all purposes.

Sequence Listing XML file XGao-10-XMLSequence.XML, created on Oct. 17, 2023 6:33 PM, size 5 kb, is incorporated by reference.

### DESCRIPTION OF RELATED ART

The present application relates to treatment of coronavirus SARS-CoV-2 infection and more particularly to a gold compound that both inhibits the replication of SARS-CoV-2 and suppresses the cytokine storm syndromes.

Note that the points discussed below may reflect the hindsight gained from the disclosed inventions, and are not necessarily admitted being prior art.

Coronavirus diseases, especially COVID-19, are infectious diseases that have spread worldwide, and have caused great damage to the economies of the world. It is commonly recognized that severe acute respiratory syndrome coronavirus 2 (SARS-CoV-2) is the cause of COVID-19. Symptoms of COVID-19 include high fever, cough, trouble breathing, persistent pain or pressure in the chest, new confusion, inability to wake or stay awake, bluish lips or face, and organ failure. It is believed that many of the symptoms are caused by cytokine storm phenomena during which multiple cytokines, such as TNF-α, IL-1, IL-6, IL-12, IFN-α, IFN-β, IFN-γ, MCP-1, and IL-8, are rapidly and massively produced in the body fluid when the body's immune system is over-activated by the virus infection. Cytokine storm has been an important cause for acute respiratory distress syndrome and multiple organ failures. Once a cytokine storm occurs, it can quickly cause single or multiple organ failure, and becomes eventually life-threatening.

Many studies have been conducted in searching for cures of COVID-19. Current drug design strategies focus either on identifying antibodies or chemical drugs that can either inhibit virus replication or suppress inflammatory cytokines. Generally, chemical drugs are of unique advantages in dealing with the COVID-19 pandemic: chemical drugs can be easily produced in large scale at low cost, thus can satisfy the treatment demands for large number of COVID-19 patients in short time. More importantly, chemical drugs allow for efficient handling, storing and distributing to patients living in environments unsuitable for biological agents. However, despite the tremendous global efforts in identifying suitable chemical drugs for the treatment of COVID-19, no chemical drug has been demonstrated to be able to treat COVID-19 infections effectively. For example, the FDA approved drug REMDESIVIR was shown to only inhibit SARS-CoV-2 replication but failed to protect lungs from inflammation injury in patients with severe infections. On the other hand, RUXOLITINIB and ACALABRUTINIB were shown to suppress inflammatory cytokine spikes, but failed to inhibit virus replication.

It would be more effective and desirable a medicine if an active gradient chemical compound can both inhibit virus replication and suppress cytokine storms. In this application such novel therapeutic agents for potentially treatment of COVID-19 are identified.

### SUMMARY

Gold compounds have been used for the treatment of rheumatoid arthritis (RA) through both oral route and parenteral route. Among them, AURURANOFIN and aurothioglucose (also known as gold thioglucose), sodium aurothiomalate (gold sodium thiomalate) are FDA approved drugs, all inhibit inflammatory cytokines associated with rheumatoid arthritis, but they have side effects that can be severe for some patients. Recently, the present inventor in U.S. Pat. No. 9,993,562 (entirety of which is hereby incorporated by reference) demonstrated that gold cluster compounds were able to achieve therapeutic outcomes that are comparable to AURURANOFIN but were without some of AURURANOFIN's side effects.

The present application further shows that gold compounds for rheumatoid arthritis treatment can also counter lung inflammations in COVID-19 animals. In addition, the gold compounds AURANOFIN (AF) and gold cluster GA are shown to form Au—S bonds in the active pockets of the viral cysteine protease M^{pro}. Because the viral 3-chymotrypsin-like cysteine protease (3CLpro, also called M^{pro}) enzyme controls the coronavirus replication and it is essential for its life cycle, M^{pro} has been a drug discovery target in seeking a treatment for COVID-19. In GA treated COVID-19 mice, not only were the replications of SARS-CoV-2 significantly reduced, but also were the inflammatory cytokines of IL-6, IL-1β, TNF-α suppressed, and no significant side effects were observed even at relatively high dosages.

For treatment of COVID-19, in one embodiment, sufficient amount of liquid suspension of gold compound molecules are orally administered to COVID-19 patients to reduce the symptoms of COVID-19.

In another embodiment, sufficient amount of gold compound molecules are administered to COVID-19 patients via parenteral injection to reduce the symptoms of COVID-19.

### BRIEF DESCRIPTION OF THE DRAWINGS

The disclosed application will be described with reference to the accompanying drawings, which show important sample embodiments of the invention and which are incorporated in the specification hereof by reference, wherein:

FIG. 1A illustrates the gold cluster structure of a GA molecule.

FIG. 1B illustrates the structure of an AURANOFIN (AF) molecule.

FIG. 1C illustrates a coronavirus genome organization, replicase gene expression and polyprotein processing.

FIG. 2A is the SDS-PAGE gel of purified M^{pro}-His tag protein and untagged SARS-CoV-2 M^{pro} in accordance with this application.

FIG. 2B is a size-exclusion chromatography of untagged M^{pro} protease in accordance with this application.

FIG. 3 is the mass spectrum of the purified M^{pro} protease in accordance with this application.

FIG. 4 is an X-ray crystal structure model of a gold compound treated M^{pro} in accordance with this application.

FIG. 5 is a cartoon presentation of an X-ray crystal structure model of gold-S bonding in Domain I-III of an M^{pro} monomer. The enlarged views are of the Au(I)—S bond sites.

FIG. 6A shows the DFT models of Au binding pockets of Cys-145 and Cys-156 of M^{pro} in the presence of Au (I).

FIG. 6B shows the DFT models of geometrically relaxed structures of the binding pockets of M^{pro.} encapsulating Au atoms, all Au—N atomic distances within 5 Å are labeled with corresponding distances, C, N, O, S and Au atoms are displayed.

FIG. 7A shows the surface presentation comparison between the catalytic pocket Cys-145 of M^{pro} and the Au(I)—S bound state with the surrounding residues shown in sticks.

FIG. 7B shows the surface presentation comparison between the catalytic pocket Cys-156 of M^{pro} and the Au(I)—S bound state with the surrounding residues shown in sticks.

FIG. 8A shows the inhibition of M^{Pro} protease activity (Panels A and C) by AF.

FIG. 8B shows the inhibition of M^{pro} protease activity (Panels B and D) by GA.

FIG. 8C shows the inhibition of M^{pro} protease activity by REMDESIVIR.

FIG. 9A shows the purified M^{pro} protease activity expressed in GA treated HEK293F cells.

FIG. 9B shows the amount of gold contained in the purified M^{pro} protease from HEK293F cells treated with GA.

FIG. 10 shows the comparison of effects on cell viability on different cell lines by AF and GA respectively.

FIG. 11A shows the western blots of different cytokine expressions in macrophages RAW 264.7 cells with or without the presence of various concentrations of GA and FA gold compounds in accordance with this application.

FIG. 11B shows the western blots of different cytokine expressions in human bronchial epithelial cell line 16HBE cells with or without the presence of various concentrations of GA and FA gold compounds in accordance with this application.

FIG. 12A shows the quantitative analysis of different cytokine expressions in macrophages RAW 264.7 cells with or without the presence of various concentrations of GA and FA gold compounds in accordance with this application.

FIG. 12B shows the quantitative analysis of different cytokine expressions in human bronchial epithelial cell line 16HBE cells with or without the presence of various concentrations of GA and FA gold compounds in accordance with this application.

FIG. 13 shows the changes of body weight, RNA viral copies and the pathology of the lungs of COVID-19 mice with or without the treatment of GA gold compound.

FIG. 14 shows the hematoxylin-eosin (HE) staining of the lungs from COVID-19 mice with or without the treatment of GA gold compound.

FIG. 15 shows the comparison of the fluorescence images stained for IL-6, IL-1β, TNF-α, and SARS-CoV-2 spike in the lungs of four COVID-19 mice in each group with or without the treatment of GA gold compound.

FIG. 16 shows the quantitative measurement of the various cytokines in the lungs of COVID-19 mice with or without the treatment of GA gold compound.

FIG. 17A shows the histopathological staining images of the lung tissue sections of normal BALB/c mice.

FIG. 17B shows the histopathological staining images of the lung tissue sections of COVID-19 BALB/c mice with NS (normal saline, 0.9% NaCl) treatment.

FIG. 17C shows the histopathological staining images of the lung tissue sections of COVID-19 BALB/c mice with GA gold compound treatment.

FIG. 18A and 18B show histopathological staining of various tissues of normal mice with or without GA treatment.

### DETAILED DESCRIPTION OF SAMPLE EMBODIMENTS

The numerous innovative teachings of the present application will be described with particular reference to presently preferred embodiments (by way of example, and not of limitation). The present application describes several embodiments, and none of the statements below should be taken as limiting the claims generally.

For simplicity and clarity of illustration, the following figures illustrate the general manner of construction, and description and details of well-known features and techniques that may be omitted to avoid unnecessarily obscuring the invention. Additionally, elements in the figures are not necessarily drawn to scale, some areas or elements may be expanded to help improve understanding of the embodiments of the invention.

The terms “first,” “second,” “third,” “fourth,” and the like in the description and the claims, if any, may be used for distinguishing between similar elements and not necessarily for describing a particular sequential or chronological order. It is to be understood that the terms used are interchangeable. Furthermore, the terms “comprise,” “include,” “have,” and any variations thereof, are intended to cover non-exclusive inclusions, such that a process, method, article, apparatus, or composition that comprises a list of elements is not necessarily limited to those elements but may include other elements not expressly listed or inherent to such process, method, article, apparatus, or composition.

The term “COVID-19” refers to the infectious disease that is at least RT-qPCR tested positive for the presence of severe acute respiratory syndrome coronavirus 2 (SARS-CoV-2). COVID-19 symptoms may include high fever, cough, trouble breathing, persistent pain or pressure in the chest, new confusion, inability to wake or stay awake, bluish lips or face, and/or organ failure. Coronaviruses are a family of enveloped, positive-strand RNA viruses responsible for a wide range of diseases in a diverse range of animal hosts. Seven human coronaviruses (HCoVs) have been identified to cause respiratory diseases of varying severities: HCoV-OC43, HCoV-229E, HCoV-NL63, HCoVHKU1, SARS-CoV, MERS-CoV and SARS-CoV-2. Among these seven HCoVs, four (HCoV-OC43, HCoV-229E. HCoV-NL63 and HCoV-HKU1) are common co-circulating. seasonal coronaviruses that cause mild respiratory tract infections generally associated with cases of the common cold. Unlike other coronaviruses, SARS-CoV-2 disease can specifically cause systemic inflammation which can develop further into acute cardiac injuries, sepsis, abnormal organ functions and heart failure. Other distinctive clinical features of SARS-CoV-2 include sore throat, hypoxaemia, dyspnoea, sneezing and diarrhoea.

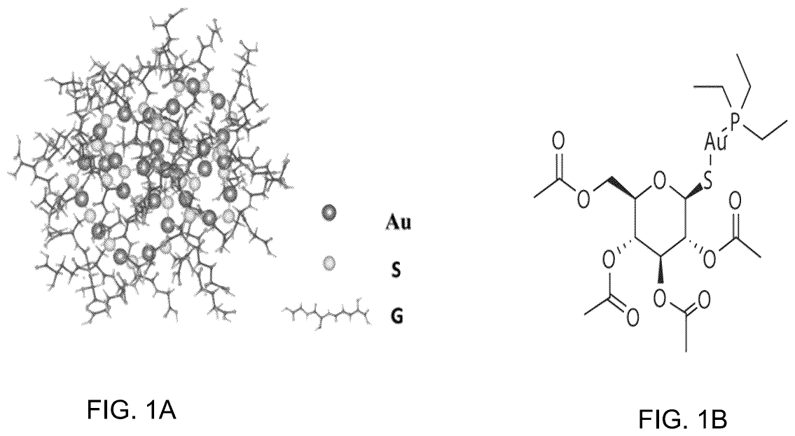

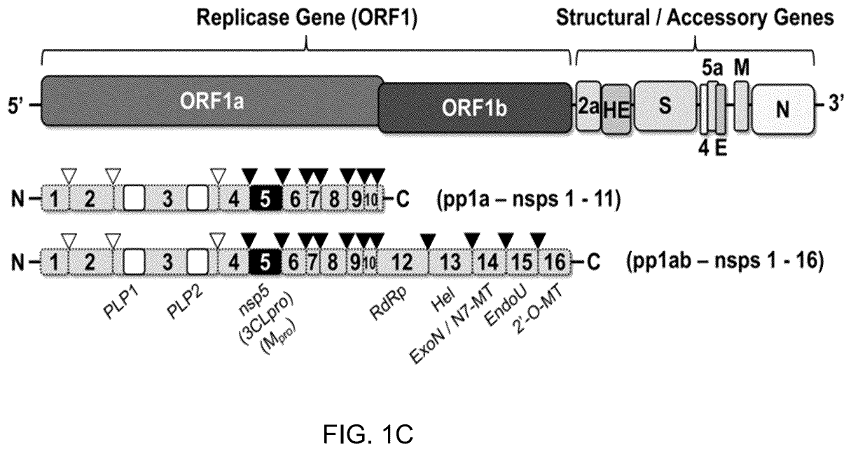

The term “gold cluster compound” refers to gold-atom cluster molecules comprised of a defined number of multiple gold atoms in a structured complex with a defined number of multiple gold binding molecules that have a thiol or selenol or phosphine or amine or arginine group, and such gold cluster molecules can characteristically emit fluorescent lights under excitation lights. The method of making of such “gold cluster” molecules is described in US 8383919 B2 to Xueyun Gao, and the entirety of which is hereby incorporated by reference. These gold cluster compounds have been found to have a variety of biological effects. For example, gold-cluster molecules were found to cause the apoptosis of cancer cells, see e. g. US 9090660 B2 to Xueyun Gao, the entirety of which is hereby incorporated by reference; to mitigate bone loss in animals, see e. g. US 10029019 B1 to Xueyun Gao, the entirety of which is hereby incorporated by reference; to mitigate rheumatoid arthritis symptoms for rheumatoid arthritic animals, see e. g. US 9993562 B2 to Xueyun Gao. In this application, gold cluster compound GA refers to the gold cluster molecule having a molecule formula Au₂₉(SG)₂₇ with a measured molecular weight of 13,983 Da by electrospray ionization mass spectrometry, and it comprises of 29 Au atoms and 27 glutathione molecules formed in a cluster structure, for illustration purpose, as illustrated in FIG. 1A. Whereas glutathion is a short peptide having a three amino acid composition of Glu-Cys-Gly (SEQ ID NO: 1). GA is this application is found to inhibit SARS-CoV-2 main protease activity both in vitro and in vivo.

The term AURANOFIN (AF) is a prescribed medicine drug refers to the gold salt having a molecule formula C₂₀H₃₄AuO₉PS⁰ with a structure illustrated in FIG. 1B. AF is an FDA approved drug for the treatment of rheumatoid arthritis and is classified by the World Health Organization as an anti-rheumatic agent. AF in this application is found to inhibit SARS-CoV-2 main protease activity both in vitro and in vivo. Another rheumatoid arthritis gold compound aurothioglucose was also found to able to inhibit M^{pro} protease activities (data not shown).

The term “SARS-CoV-2” strain used in this research was isolated from a COVID-19 patient (BetaCoV/Wuhan/IVDC/-HB-01/2020, EPI_ISL_402119) that are passaged on Vero cells. In particular, the viral RNA was extracted from 100 μL supernatant of infected cells using the automated nucleic acid extraction system (TIANLONG, China) by following the manufacturer's recommendations. For SARS-CoV-2 virus detection was performed using the One Step PrimeScript RT-PCR kit (TAKARA, Japan) on the LIGHTCYCLER 480 REAL-TIME PCR system (Roche, Rotkreuz, Switzerland). The replicase gene of SARS-CoV-2 was used for the detection which comprises two open reading frames, ORF1a and ORF1ab. The primers targeting SARS-COV-2 ORF1a were used in the study: Forward primer (SEQ ID NO. 2):5′-AGAAGATTGGTTAGATGATGATAGT-3′; Reverse primer (SEQ ID NO. 3):5′-TTCCATCTCTAATTGAGGTTGAACC-3′; Probe (SEQ ID NO. 4):5′-FAM-TCCTCACTGCCGTCTTGTTGACCA-BHQ1-3′.

The term “M^{pro}” refers to SARS-CoV-2 main protease, is the papain-like protease(s) responsible for processing the polyproteins that are translated from SARS-CoV-2 viral RNA. “M^{pro}” is also called coronavirus protease nsp5 or 3CL^{pro} that is an approximately 30 kDa, three-domain cysteine protease conserved in structure and function in all known coronaviruses and serves as the main protease for proteolytic processing of the replicase polyproteins (pp1a and pp1ab). Typically, coronaviruses code for two or three proteases to process the replicase polyprotein: one or two papain-like proteases (PLPs) encoded within nsp3, and one main protease, nsp5 (3CL^{pro} or M^{pro}). PLPs are responsible for cleavage events between nsp1 and the N terminus of nsp4, whereas all remaining pp1a/pp1ab cleavage events are mediated by nsp5. The name ‘main protease’, or M^{pro}, refers to the critical role of this protease in coronavirus gene expression and replicase processing, and its other name 3C-like protease (3CL^{pro}) refers to the similarities between this protease and 3C proteases seen in picornaviruses, namely their similar substrate specificities and core structural homology. Among coronaviruses, nsp5 proteases within the same genus generally exhibit sequence identity of greater than 80% whereas protease in different genera are far more divergent with sequence identity much closer to 50% despite high tertiary and quaternary structural conservation especially in domains 1 and 2. Sequence analysis of the SARS-CoV and SARS-CoV-2 proteases reveals only 12 residue differences (approximately 96% identity) spread throughout the structure of the protease, with the majority of these residues distant from the active site (including along the distal surface of domain 1 and within domain 3), which strongly supports the prospect of developing active-site inhibitors that target both proteases.

FIG. 1C shows the coronavirus open reading frames (ORFs) associated with replication (replicase gene; ORF1a/ORF1b) and the structural and accessory genes. The two variant polyproteins (pp1a and pp1ab) translated from the replicase gene are shown with the non-structural protein domains of the polyprotein labelled and the proteolytic cleavage sites marked with arrows. Three proteases mediate the proteolytic processing of the replicase polyproteins (PLP1, PLP2 and nsp5 (3CL^{pro}/M^{pro})), and white arrows for PLPs and black arrow for nsp5 for each cleavage site correspond to the protease responsible for mediating its cleavage, wherein PLP stands for papain-like protease; RdRp stands for RNA-dependent RNA polymerase; Hel stands for helicase; ExoN stands for exonuclease; N7-MT stands for N7-methyltransferase; EndoU stands for endoribonuclease; 2′-O-MT stands for 2′-O-methyltransferase.

Typically, immediately upon entry into a host cell, the virus translates its replicase gene (ORF1) which consists of two large, overlapping ORFs, ORF1a and ORF1ab. Located at the end of ORF1a, a ribosome frame-shifting sequence consisting of an RNA pseudoknot causes the co-translation of two large polyprotein precursors of differing lengths, pp1a and pp1ab. Polyprotein pp1a contains non-structural proteins (nsps) 1-11, and polyprotein pp1ab comprises the complete translated coding region of nsps 1-16. Essential for virus replication is the proteolytic processing of these polyproteins by virus-encoded proteases to yield the mature and functionally active replication machinery of the virus. Once proteolytically processed, the translation products of pp1a collectively modulate host cell factors and help prepare the cell for viral RNA synthesis through the formation of replication complexes, while the C-terminal translation products of pp1ab largely catalyse and/or regulate the processes of RNA replication and transcription driven by the viral RdRp (nsp12). Replication complexes assemble on virus-induced membrane structures such as double-membrane vesicles and convoluted membranes driven by transmembrane nsps 3, 4 and 6. The active replication complex promotes the continuous and discontinuous synthesis of negativesense RNA templates, which are subsequently used to drive formation of genomic copies and a nested set of subgenomic RNAs from the downstream ORFs encoding structural and accessory proteins, respectively. Following replication of genomic and subgenomic RNA on double-membraned vesicles, structural proteins like the spike (S), envelope (E), matrix (M) and nucleocapsid (N) proteins are translated by existing positive-strand subgenomic RNAs. S, E and M become glycosylated within the Golgi before localizing to the endoplasmic reticulum-Golgi intermediate compartment (ERGIC) to be assembled into virions.

Proteolytic processing acts as a key regulatory mechanism in the expression of the coronavirus replicase proteins, blocking this process has been demonstrated to block viral replication entirely. Background information can be found in Roe et al., “Targeting novel structural and functional features of coronavirus protease nsp5 (3CLpro, Mpro) in the age of COVID-19,” Journal of General Virology, PMID, 33507143, DOI 10.1099/jgv.0.001558 (Jan. 28, 2021), the entirety of which is therefore incorporated by reference.

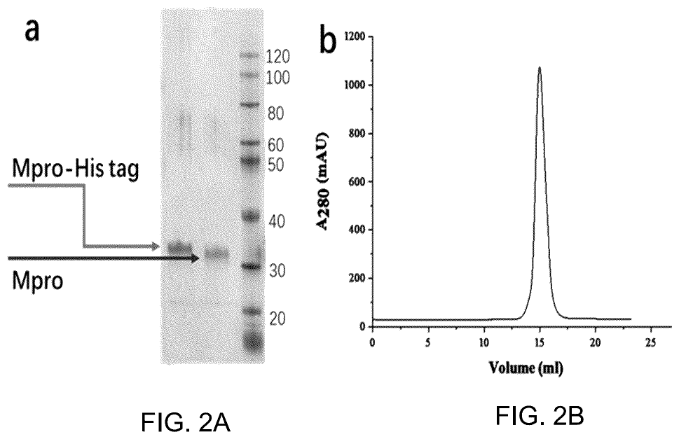

In the present application purified SARS-CoV-2 M^{pro} protein was obtained through cloning and expression of M^{pro} gene in E. coil. The full-length gene encoding SARS-CoV-2 M^{Pro} was optimized and synthesized for Escherichia coli (E. coil) expression through GENEWIZ system. The gene was cloned into a modified pET-28a expression vector with an N-terminal (His)6-tag followed by a Tobacco etch virus (TEV) cleavage site. The construct was confirmed by DNA sequencing. The plasmid was further isolated and transformed into the Escherichia coli Rosetta (DE3) expression strain of Invitrogen Inc. The E. coli cells containing the plasmids above were grown to an OD600 of 0.8 and induced with isopropyl b-D-thiogalactopyranoside (IPTG) to a final concentration of 0.5 mM at 16° C. for 14 hrs. E. coli cells were then harvested by centrifugation at 4600 g, re-suspended in lysis buffer (120 mM Tris/HCl, pH 8.0, 20 mM imidazole and 300 mM NaCl) and lysed by French press, the lysate was centrifuged at 15 000 g for 50 min. Then the supernatant was loaded on to a Ni-NTA column pre-equilibrated with lysis buffer, and washed with 20 mM Tris/HCl, pH 8.0, 300 mM NaCl and 50 mM imidazole. The protein was eluted in 20 mM Tris/HCI, pH 8.0, 150 mM NaCl and 300 mM imidazole. TEV protease was added to the His tag fused protein solution and dialyzed overnight into anion-exchange chromatography buffer A (20 mM Tris/HCl, pH8.0, 20 mM NaCl, 1 mM DTT, 1 mM EDTA) to cleave off the His(6) tag. The tag cleaved protein was further purified using a Resource-Q column of AKTA fast protein liquid chromatography of GE Healthcare, Inc by elution with a linear gradient of 20-500 mM NaCl, 20 mM Tris/HCl, 1 mM EDTA, 1 mM DTT and pH 8.0. The purity of the obtained Mpro was analyzed by SDS/PAGE at each step. The purified and concentrated SARS-CoV-2 M^{pro} was stored in 20 mM Tris-HCl (pH 7.3), 20 mM NaCl, 1 mM DTT, 1 mM EDTA for enzyme activity assays and crystallization. FIG. 2A shows the SDS/PAGE gel electrophoresis of the purified M^{pro} protein and FIG. 2B shows the size-exclusion chromatography profile of M^{pro} after the cleavage of the His(6) tag.

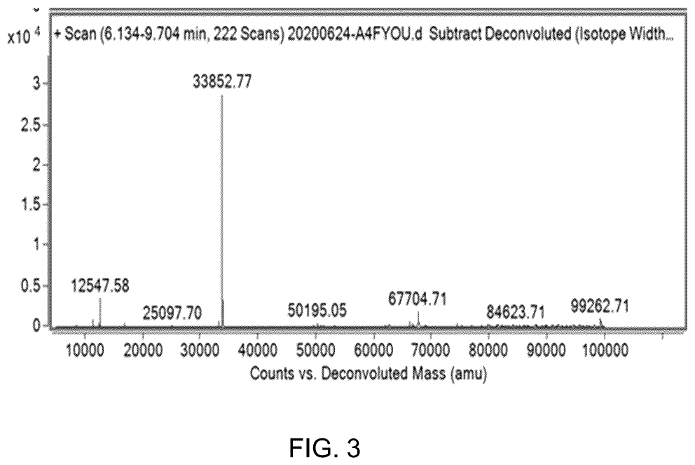

Five μl of the purified M^{pro} protein was used for Liquid chromatography-mass spectrometry analyses in positive-ion mode with a quadrupole-time-of-flight mass spectrometer combined with a high-performance liquid chromatograph for detecting the molecular weight of of the purified MPTO protein. Mass deconvolution was performed using AGILENT MASSHUNTER Qualitative Analysis B.06.00 software with BIOCONFIRM WORKFLOW. The purified M^{pro} protein mass spectroscopy profile is shown in FIG. 3.

The purified M^{pro} protein was used for crystallization at 22° C. using the sitting-drop vapor-diffusion technique. About 0.7 μl 6 mg/ml protein solution mixed with an equal volume of reservoir solution was used for growing crystals. Initial crystals were found under the crystallization conditions of the PEG/Ion Screen Kit of CRYSTAL SCREEN by Hampton Research. After optimization, the best crystals of M^{pro} protein were obtained under the condition of 200 mM KF and 15% PEG 3350 after 4 to 5 days. For gold compound treatment, crystals of M^{pro} proteins were further soaked in reservoir solutions in the presence of 10 mM GA gold cluster compound solution or 10 mM AURANOFIN compound solution for over 15 hr. M^{pro} crystals treated with GA or AF were then X-ray analyzed for structure changes.

Prior to data collection, all crystals were cryo-protected by plunging them into a drop of reservoir solution supplemented with 10-20% glycerol, then flash frozen in liquid nitrogen. The X-ray diffraction data were collected at the beamlines in Shanghai Synchrotron Radiation Facility and were processed using software HKL3000 or XDS. The initial phase was determined by molecular replacement method using the program PHASER from CCP4 program suit, with the crystal structure of SARS-CoV-2 main protease M^{pro} in complex with an inhibitor N3 (PDB entry 6LU7) as the initial model. The structure refinement was carried out using PHENIX and REFMAC, model building was carried out by COOT, and MOLPROBITY was used to validate the structure. The locations of Au (I) ions were identified according to the anomalous difference Fourier maps. Data collection and refinement statistics are listed in Table I. M^{pro} crystal structures were constructed using PYMOL as available at pymol.org.

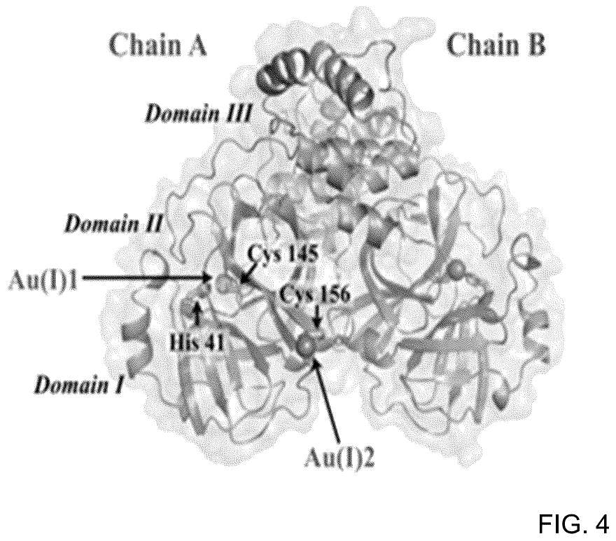

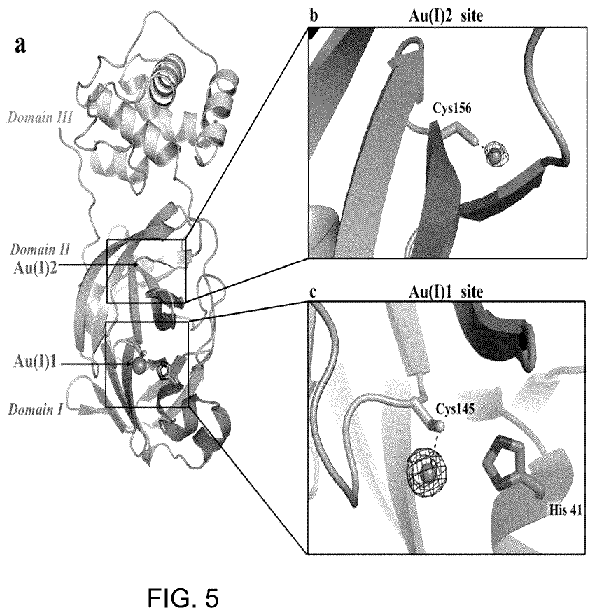

In reference to FIG. 4, the resulting SARS-CoV-2 M^{pro} crystal structures in the presence either gold compounds (GA or AF) were very similar and shared most of the features as those of the crystal structures of the native M^{pro} previously determined. However, the densities of two Au(I) ions were found near the thiol residues of Cys145 and Cys156 of M^{pro} after the M^{pro} crystals were treated with either GA or AF compounds. The positions of these two Au(I) ions were confirmed by applying the anomalous difference Fourier maps, as shown in FIG. 5.

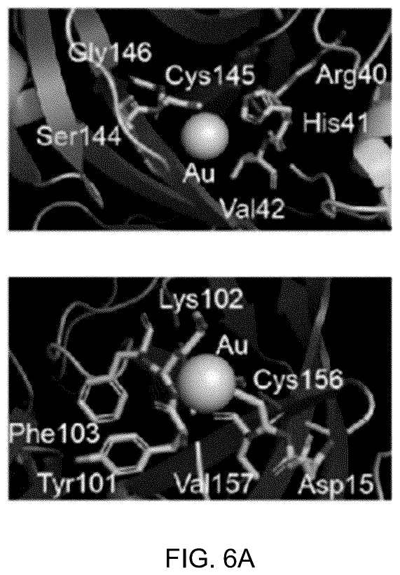

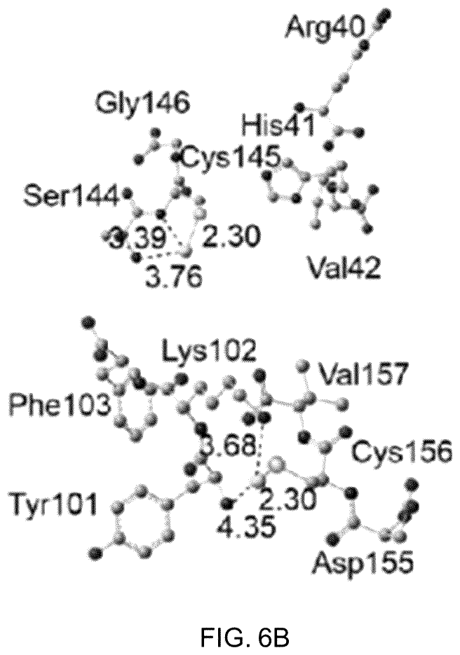

The binding energies between Au(I) and M^{pro} protein were calculated by density functional theory (DFT) calculations. According to the crystal structure shown in FIG. 4. M^{pro} protein has two binding pockets for Au(I) ion and each encapsulates one Au atom. The two Au atoms are binding with the S atoms of Cys145 and Cys156 respectively. To simulate the chemical environment of the Au atoms bound in the binding pockets, the residues within 5 Å from the Au atoms were considered. Specifically, Ser144, Cys145, Gly 146, Arg40, His41, and Val42 in the first pocket, and Tyr101, Lys102, Phe103, Asp155, Cys156 and Val157 in the second pocket were used for calculation. There are four peptide bonds in each pocket. To maintain the skeleton structures of the two pockets, positions of C and N atoms of the peptide bonds were fixed and all the other atomic positions were allowed to be relaxed during the geometry optimizations. FIG. 6A and FIG. 6B. The B3LYP functional in conjunction with the SDD basis set for Au and the 6-31G (d, p) for nonmetal atoms were applied. The SDD pseudopotential was also applied for Au. During geometry optimizations, SMD solvation model was utilized to model the water environment. All the calculations were carried out using GAUSSIAN 09 package. The bond dissociation energy (EBD) between Au and the protein binding pockets was calculated using the following equation,

$$EBD = {E}_{Au} + {E}_{ligands} - {E}_{complex} \qquad ( 1 )$$

where E_{Au}, E_{ligands}, and E_{complex} were the total energies of Au atom, ligands of the pocket, and the complex, respectively. E_{ligands} was obtained by single-point energy calculation based on the optimized geometries of complexes with Au atom removed. DFT calculations confirmed that Au atoms preferred to form S—Au bond with the thiol groups of Cys145 and Cys156 of the M^{pro} protein binding pockets. In addition, the N atoms of Ser144 and Cys145 and those of Tyr101 and Lys102 have a distance within 5 Å from the corresponding Au atoms, suggesting there may be considerable electrostatic interactions between the respective N atoms and Au atoms. The bond dissociation energies (EBD's) between Au and the two pockets are calculated to be respectively 46.1 kcal mol⁻¹ and 26.5 kcal mol⁻¹. Such large EBDs suggest that the respective Au atoms are firmly locked inside the pockets, which can cause the effective inhibition of the proteinase activity of M^{pro}.

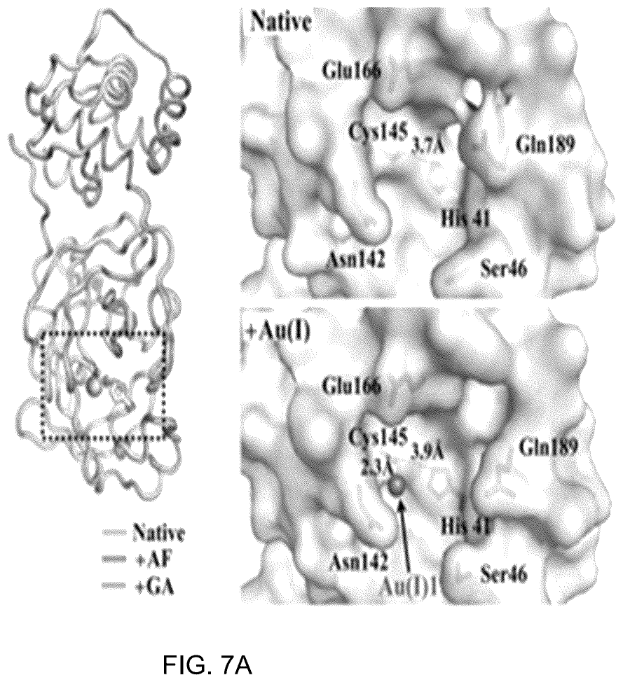

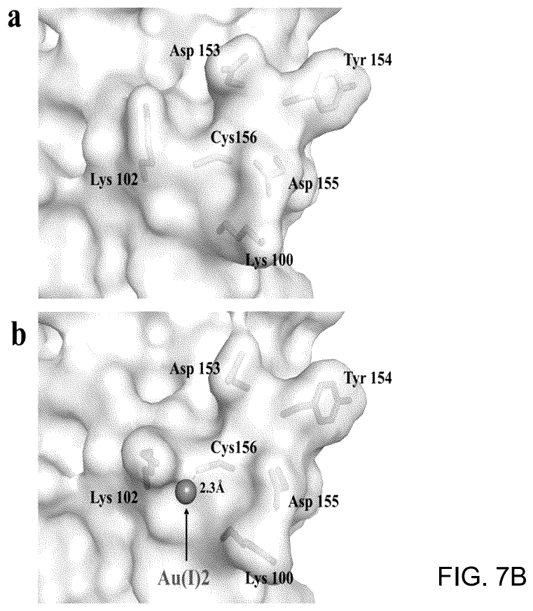

In references to FIG. 7A and 7B, the Au(I)-Cys145 and Au(I)-Cys156 interactions are illustrated by superpositions of the crystal structures of AF treated M^{pro}, GA treated M^{pro} and the untreated M^{pro}. The surface presentations and the surrounding residues of the catalytic sites of native and Au(I)—S bound M^{pro} are shown to form binding pockets.

TABLE 1
Crystal X-ray Data collection and refinement statistics

| | M^{pro}-AF | M^{pro}-GA | |
|---|---|---|---|
| | treated | treated | M^{pro}-Native |
| Data collection | | | |
| Wavelength (Å) | 0.86 | 0.98 | 0.98 |
| Space group | C2 | C2 | C2 |
| Cell | | | |
| dimensions | | | |
| a, b, c (Å) | 114.3, | 113.8, | 113.9, 53.8, |
| | 54.0, 44.7 | 53.8, 44.6 | 44.7 |
| α, β, γ (°) | 90.0, | 90.0, | 90.0, 101.5, |
| | 101.8, 90.0 | 102.0, 90.0 | 90.0 |
| Resolution (Å) | 50-2.75 | 50-1.72 | 50-1.77 |
| | (2.90-2.75) ^{a} | (1.75-1.72) ^{a} | (1.80-1.77) ^{a} |
| R_{merge} | 0.094 | 0.089 | 0.070 (0.542) |
| | (0.154) | (0.823) | |
| [symbol] I/σ(I) [symbol] | 14.0 (7.0) | 33.8 (2.2) | 24.3 (2.4) |
| Completeness | 96.4 | 99.8 | 99.3 (95.9) |
| (%) | (80.9) | (100.0) | |
| Redundancy | 5.4 (3.4) | 6.2 (6.0) | 6.6 (4.9) |
| Refinement | | | |
| Resolution (Å) | 50-2.75 | 50-1.72 | 50-1.77 |
| No. reflections | 6,781 | 27,877 | 25,365 |
| R_{work/}R_{free} | 0.194/0.228 | 0.199/0.237 | 0.206/0.248 |
| No. atoms | | | |
| Protein | 2,329 | 2,329 | 2,329 |
| Au | 2 | 2 | 0 |
| Water | 110 | 234 | 260 |
| B-factors | 34.8 | 37.8 | 24.7 |
| Rmsd bond | 0.007 | 0.009 | 0.008 |
| length (Å) | | | |
| Rmsd bond | 1.0 | 1.0 | 1.0 |
| angle (°) | | | |
| Ramachandran | | | |
| Plot | | | |
| Favored (%) | 97.7 | 97.7 | 99.0 |
| Allowed (%) | 2.3 | 2.3 | 0.7 |
| Outliers (%) | 0 | 0 | 0.3 |

^{a} The values in parenthesis mean those of the highest resolution shell.

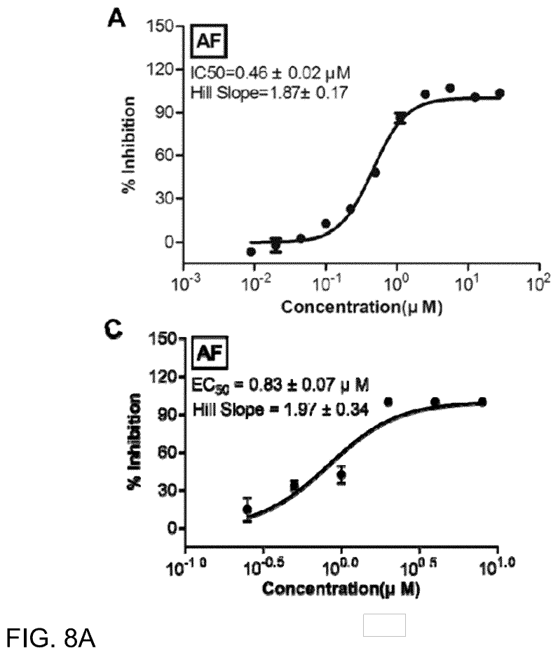

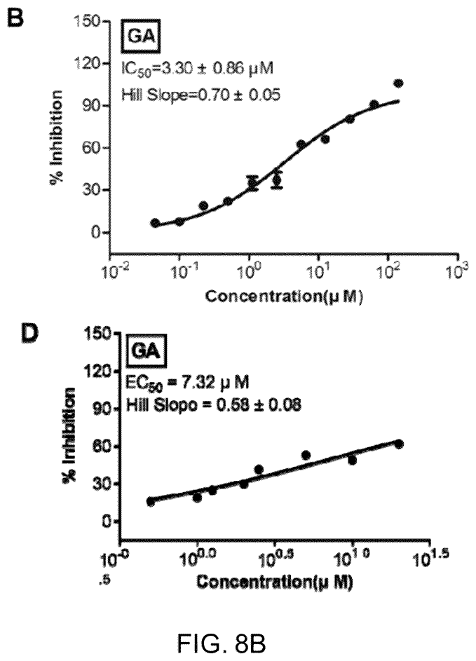

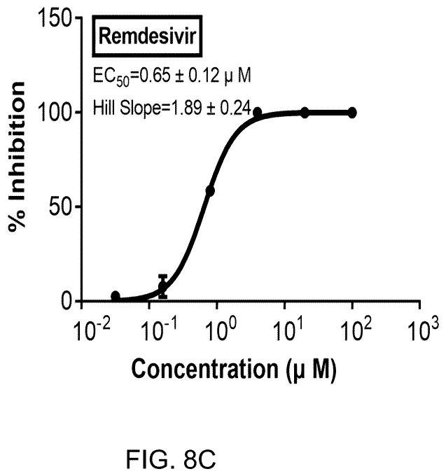

In reference to FIG. 8A and 8B, the inhibitory effects of GA and AF on protease activities of M^{pro} were measured according to the method by V. Grum-Tokars, et al., “Evaluating the 3C-like protease activity of SARS-Coronavirus: Recommendations for standardized assays for drug discovery,” Virus Research 133, 63-73 (2008), the entirety of which is therefore incorporated by reference. Eleven Different concentrations of GA or AF were added into M^{pro} protease reaction mixture that contained 0.5 μM M^{pro} protein, 20 μM substrate (SEQ ID No: 5) (EDNAS-Glu)-Ser-Ala-Thr-Leu-Gln-Ser-Gly-Leu-Ala-(Lys-DABCYL)-Ser. M^{pro} activity was measured by fluorescence resonance energy transfer (FRET) assay. Fluorescence intensity was monitored by the multimode plate reader from Bio-Rad with excitation at 340 nm and emission at 535 nm. All experiments were performed in triplicates. The fluorescence labeled substrate, (SEQ ID No: 5) (EDNAS-Glu)-Ser-Ala-Thr-Leu-Gln-Ser-Gly-Leu-Ala-(Lys-DABCYL)-Ser, was derived from the auto-cleavage sequence of the viral protease and was chemically modified for enzyme activity assay.

EC50 measurements with SARS-CoV-2 were performed under biosafety level 3 (BSL-3) conditions at Chinese Center for Disease Control and Prevention, China. Vero cells were infected with SARS-CoV-2 at a multiplicity of infection (MOI) of 0.015 diluted in DMEM/F12 without FCS at 37° C. for 1 h. Cells were washed with DMEM/F12 with 10% FCS and supplemented with AURANOFIN or gold cluster GA in different concentrations. For solvent control, cells were only treated with 1% DMSO 48 hours after infection (h.p.i.), cells supernatant were collected and virus RNA samples were subjected to qRT-PCR measurement. All experiments were performed in triplicate.

In FIG. 8A panel A shows the IC50 curve of AF, panel B shows the EC50 of AF. The inhibition of 50% purified M^{pro} protease activity (IC50) was reached by ˜0.46 μM AF. EC50 was tested on SARS-CoV-2 replication in Vero cell in the Biosafety Level-3 Lab of China CDC, and panel B in FIG. 8A shows that the EC50 of AF was ˜0.83 μM. The testing method was according to A. Pizzorno, et al., “Characterization and treatment of SARS-CoV-2 in nasal and bronchial human airway epithelia,” Cell Rep. Med. 1, 100059 (2020), the entirety of which is incorporated by references.

Comparing to the reported IC 50 for the known COVID-19 drug EBSLEN is 0.67 μM, AURANOFIN is shown to be a strong inhibitor for M^{pro} protease activity. Whereas the EC50 for the known COVID-19 drug REMDESIVIR is ˜0.65 μM (FIG. 8C), EC50 of AURANOFIN (˜0.83 μM) is close to the effectiveness of REMDESIVIR.

Similar measurements were performed using gold cluster compound GA, as shown in FIG. 8B, panel A shows that the inhibition of 50% purified M^{pro} protease activity (IC50) was reached by ˜3.30 μM of GA and panel B shows that the inhibition of 50% SARS-CoV-2 replication in Vero cell was reached by ˜7.32 μM of GA.

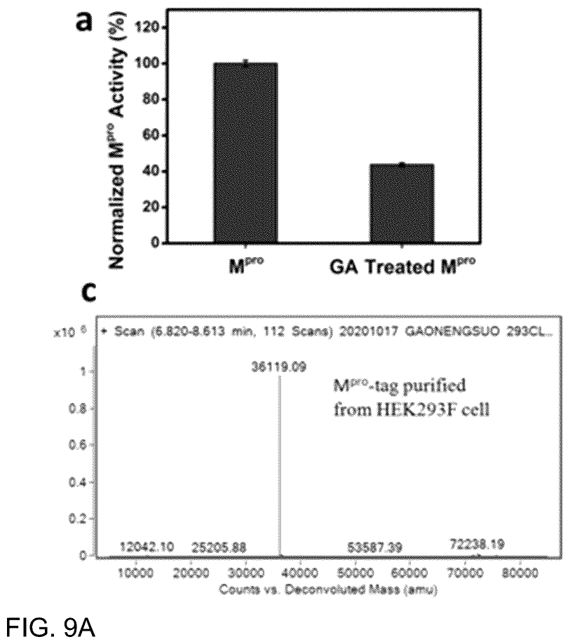

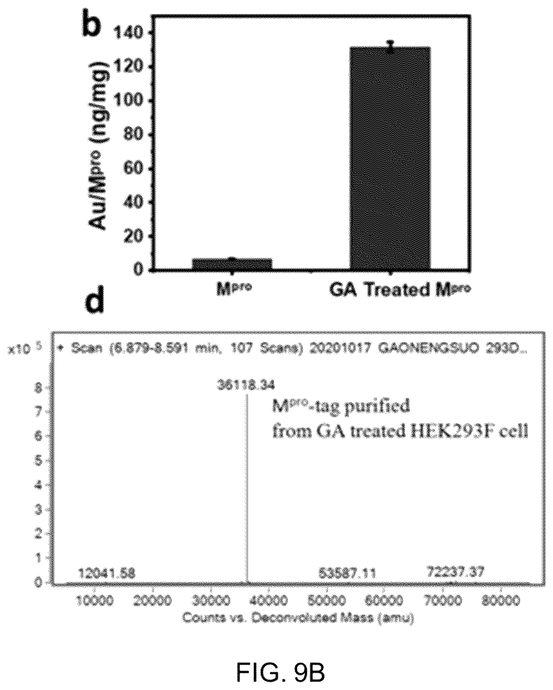

The inhibitory effect of GA on M^{pro} protease activity in vivo was further tested on HEK293F cells transiently transfected with a plasmid containing strep-tagged SARS-CoV-2 M^{pro} gene. The M^{pro} gene was expressed for 24 hrs in HEK293F cells before GA was added to the culture medium for a final concentration of 500 μM, and cells were cultured for an additional 24 hrs. After cells were harvested, SARS-CoV-2 M^{pro} proteins were extracted from GA-treated HEK293F cells and were purified and analyzed for enzyme activity and by MASS spectroscopy. As shown in FIG. 9A, in panel A, the M^{pro} protease extracted from GA treated HEK293F cells was about 40% as that of control M^{pro} activity. FIG. 9B confirms that the M^{pro} protein purified from GA-treated HEK293F cells contained gold atoms. There was about 120 ng Au per mg M^{pro} protein extract. However the mass spectroscopy of M^{pro}-tag purified from untreated HEK293F cells showed a matching molecular weight ˜36119Da, while the mass spectroscopy of M^{pro}-tag purified from GA treated HEK293F cells showed a band of molecular weight of ˜36118Da, the missing Au signal in the mass spectroscopy of M^{pro}-tag purified from GA treated HEK293F cells was probably due to the laser ablation that would have broken the Au—S bond of the samples.

Recently Rothan et al reported that AF well inhibited SARS-CoV-2 replication in infected Huh cells and the EC50 of AF was ˜1.4 μM, and they speculated that inhibition of SARS-CoV-2 replication might be induced by gold compound suppressing the thioredoxin reductase activity and inducing ER stress of host cells. See H. A. Rothan, et al, “The FDA-approved gold drug Auranofin inhibits novel coronavirus (SARS-COV-2) replication and attenuates inflammation in human cells,” Virology 547, 7-11 (2020). However, based on the crystal structure studies in this application and the M^{pro} activity data reported herein, it is more likely that the gold compounds inhibit SARS-CoV-2 replication via Au(I) binding to Cys145 and Cys 156 of M^{pro}, causing the inhibition of its activity in host cells.

To test whether GA and AF are toxic to normal cells, Vero E6, RAW264.7 and 16HBE cell line were tested. Various doses of AURANOFIN or GA gold cluster were added into cell culture media respectively. After 48 hrs incubation, cell viabilities were checked by CCK8 (Beyotime, China) following manufacturer's instruction, all studies were carried in triplicate. All cell lines were obtained from ATCC with authentication service.

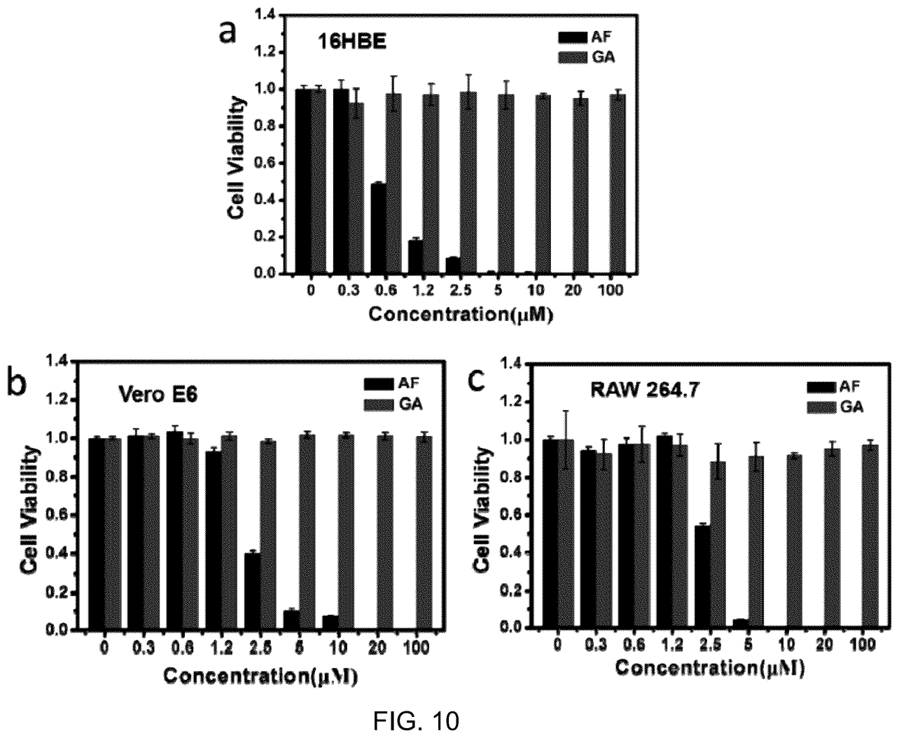

In reference to FIG. 10, for human bronchial epithelial cells (16HBE), the CC50 of AF was about ˜0.6 μM while gold cluster GA showed no cell toxicity even at a concentration of 100 μM in cell cultures for 48 hrs. For Vero E6 cell, the CC50 of AF was about ˜2.2 μM while gold cluster GA showed no cell toxicity even at 100 μM in cell cultures for 48 hrs. For macrophage RAW264.7 cells, the CC50 of AF was about ˜2.4 μM and gold cluster GA again showed no cell toxicity even when its dose was increased to 100 μM in cell cultures for 48 hrs. For in vivo toxicity, in terms of mice acute toxicity, the intraperitoneal LD50 for AF was known to be about ˜33.8 mg/kg. In terms of rat acute toxicity, the intraperitoneal LD50 for AF was known to be ˜25.5 mg/kg and it was about ˜288 mg/kg.bw for GA.

The in vivo toxicity LD50 was measured in mice as follows. 100 adult female BALB/c mice for experiments were conducted in compliance with regulations of the National Act on the use of experimental animals (China) and were approved by the Institutional animal care and ethic committee at the Chinese Academy of Sciences (approved No. SYXK (jing) 2014-0023). Their weights ranged between (18 g˜22 g). Mice were housed in plastic cages, each cage contained 10 mice. Animals were kept under controlled temperature of 25±2° C. for 12 hours under light and 12 hours dark cycle throughout the experiment. The LD50 was studied by a “staircase method” with increasing doses of FA or GA. Ten doses of 100, 200, 300, 400, 500, 600, 700, 800, 900, 1000 mg/kg b.wt per body weight., were given to 10 groups of mice (10 in each) for the determination of intraperitoneal LD50 in female mice. Animals were observed for the 2, 6, and 24 hours for any toxic symptoms. After 24 hours, numbers of died animals was counted in each group and LD50 was determined by the method of Karber. In this study, no mice were dead within 24 hour after various amounts of GA were injected.

In the rheumatoid arthritis rat/mice treatment model, when oral AF was given at a dose of 6˜9 mg/kg.bw and intraperitoneal injection of GA was given at 5 mg/kg.bw, both of the two treatments doses of AF and GA showed significant suppression of inflammatory cytokine levels and were observed to achieve a similar outcome for rheumatoid arthritis treatment. However, the toxicity data on cellular level, on mice and rats in vivo, and on rheumatoid arthritis model mice/rat treatments, all suggest that gold cluster GA is a safer gold compound, it may be a better choice as a drug and of higher safety than AF when electing them as treatments COVID19 patients.

TABLE II
Mice/Rat Acute Toxicity and Cytotoxicity of GA and AF

| | Gold compounds | |
|---|---|---|
| Animal/cell | GA | AF |
| BALB/c Mice | LD50 > 1000 | LD50~33.8 |
| | mg/kg.bw | mg/kg.bw |
| SD Rat | LD50~288 | LD50~25 |
| | mg/kg.bw | mg/kg.bw |
| epithelial cell | CC50 31.9 μM | CC50~0.63 μM |
| Vero cell | CC50 33.84 μM | CC50~2.27 μM |
| Macrophage cell | CC50 43.2 μM | CC50~2.63 μM |

Most recently, a clinical study revealed that severe COVID-19 patients have a hyperinflammatory immune response associated with macrophage activation. See Y. Cao, et al. “Ruxolitinib in treatment of severe coronavirus disease 2019 (COVID-19): A multicenter, single-blind, randomized controlled trial,” J. Allergy Clin. Immunol. 146, 137-146 (2020). By using RA treatment drugs, RUXOLITINIB, to inhibit the activation of NFKB pathway in macrophages, down regulation of the expression level of IL-6, IL-1β, TNF-α were observed and the oxygenation and clinical status of most severe patients on supplemental oxygen were improved relatively rapidly.

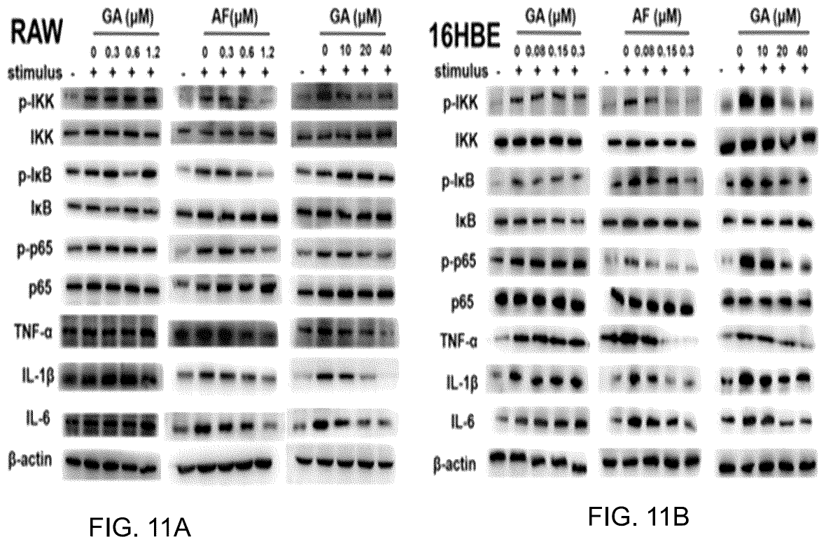

In reference to FIG. 11A and 11B, the effects of gold compound AF and GA on cytokine expressions were measured using mircophage cell lines RAW 264.7 (FIG. 11A) and human bronchial epithelial cell 16HBE cell (FIG. 11B). RAW 264.7 or 16HBE cells were seeded into 6 well plates at a density of 2 ×10⁶ cells/well. After incubation with or without the presence of TNFα (50 ng mL⁻¹) under different concentrations of AURANOFIN or gold cluster GA for 24 hrs, the cells were collected and lysed with RIPA buffer (50 mmol L⁻¹ Tris-HCl, pH 7.4, 150 mmol L⁻¹ NaCl, 1% Triton X-100, 1% sodium deoxycholate, 0.1% SDS, 1 mmol L⁻¹ sodium orthovanadate, 50 mmol L⁻¹ NaF, and 1 mmol L⁻¹ethylenediaminetetraacetic acid) along with protease inhibitor purchased from Roche Molecular Biochemicals. The collected cell lysates were centrifuged at 13000 rpm for 10 min, and the supernatants were stored for the subsequent Western Blot analysis. The protein concentrations of the supernatants were determined using a microplate spectrophotometer (SPECTRAMAX M4 of Molecular Devices, USA) at a wavelength of 595 nm. An equal quantity of proteins (50 μg) were separated by running on 10% SDS-PAGE and the separated proteins were transferred to a polyvinylidene fluoride (PVDF) membrane (0.45 μm, Millipore, USA). After blocking, the membranes were incubated with specific antibodies for COX-2 (from Cell Signaling Technologies, 12282, 1:1000), IL-1β (from Cell Signaling Technologies, 12703, 1:1000), IL-6 (from Cell Signaling Technologies, 12912, 1:1000), TNF-α (from Cell Signaling Technologies, 11948, 1:1000), phosphor-p65 (from Cell Signaling Technologies, 3033, 1:1000), p65 (from Cell Signaling Technologies, 3034, 1:1000), phosphor-IκBα (from Cell Signaling Technologies, 2859, 1:1000), IκBα (from Cell Signaling Technologies, 4812, 1:1000), IKKα (from Cell Signaling Technologies, 2682, 1:1000), IKKβ (from Cell Signaling Technologies, 8943, 1:1000), phosphor-IKKα/β (from Cell Signaling Technologies, 2697, 1:1000), this step was followed by incubation with an appropriate secondary antibody conjugated to horseradish peroxidase (Beyotime Biotechnology, China) to produce visible fluorescence for measurement.

Shown in FIG. 11A and 11B, relatively low dose of AF (1.2 μM) significantly suppressed IL-6, IL-1β and TNF-αexpression levels in both macrophage cells and human bronchial epithelial cells. Relatively high doses of GA (40 μM) also significantly suppressed the levels of IL-6, IL-1β, TNF-α in both inflammatory macrophage cells and human bronchial epithelial cells.

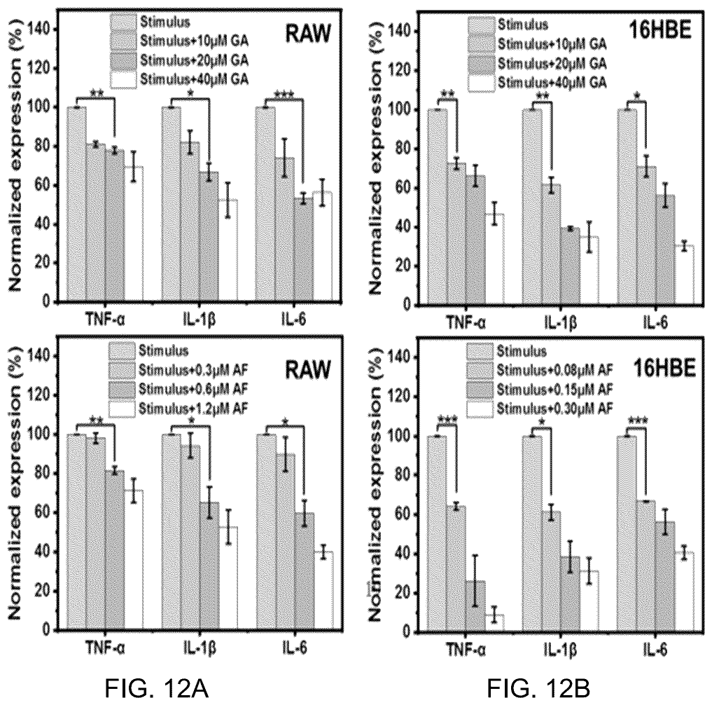

The dose-dependent cytokine expression suppressions are quantitatively shown in FIG. 12.

In inflammatory macrophages, the nuclear factor NFκB is the key signaling pathway that regulates the inflammatory mediator genes which involves the inflammatory factors to induce the activation of the IκB kinase (IKK) complex, causing subsequent degradation of IκB proteins, and releasing p-p65 which enters the nuclei to induce the expression of TNF-α, IL-1β and IL-6. As shown by the Western blots, AF or GA treatment could decrease IKK phosphorylation level, causing suppressed IκB phosphorylation and the inhibition of p65 phosphorylation. In this case, AF at low dose (1.2 μM) could inhibit phosphorylation of IKK, IκB, and p65, thus suppress the NFκB activation. GA at a relatively high dose (40 μM) could inhibit thephosphorylation of IKK, IκB, and p65, thus suppress NFκB activation in inflammatory macrophage cells and human bronchial epithelial cells.

As reported in COVID-19 patients, virus infected bronchial epithelial cell would activate NFκB pathway to express inflammatory cytokine, these cytokines would activate macrophages into inflammatory status. Shown in FIG. 11B, gold compounds were able to inactivate NFκB pathway and suppress inflammatory cytokine expression level in inflammatory human bronchial epithelial cells. AF in low dose of (0.15 μM) and GA of high dose (20 μM) may have significantly inhibited the phosphorylation level of IKK, IκB, p65 to suppress NFκB activation, thus inhibited IL-6, IL-1β, and TNF-α inflammatory cytokine expression in these bronchial epithelial cells.

### GA Treatment of COVID 19 Mice

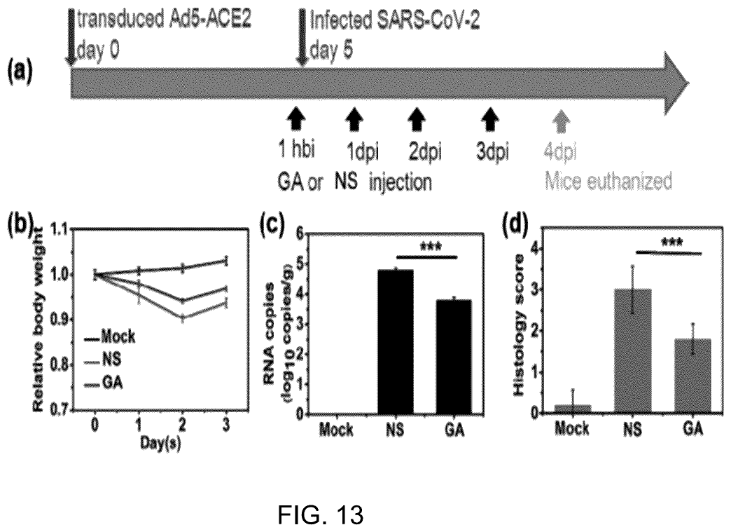

The COVID-19 mice model was generated following recently reported method and detailed procedures are illustrated in FIG. 13. See J. Sun, et al, “Generation of a broadly useful model for COVID-19 pathogenesis, vaccination, and treatment,” Cell 182, 734-743 (2020).

In particular, pathogen-free 6 week old female BALB/c mice were purchased from SiPeiFu Laboratory Animal Co (Beijing, China). All protocols were approved by the Institutional Animal Care and Use Committees of National, Institute for Viral Disease Control & Prevention, Chinese Center for Disease Control and Prevention. The SARS-CoV-2 strains used in this research were isolated from COVID-19 patient (BetaCoV/Wuhan/IVDC/-HB-01/2020, EPI_ISL_402119) and passaged on Vero cells. The human serotype 5 adenoviral vector expressing human ACE2 under the control of the CMV promoter was a gift kindly provided by Dr. Zhao Juncun.

COVID-19 mice were generated as previously reported. See S. Du, et al., “Structurally resolved SARS-CoV-2 antibody shows high efficacy in severely infected hamsters and provides a potent cocktail pairing strategy,” Cell 183, 1-11 (2020). 12 mice were divided into 3 groups with four mice each, at day 0, mice were anesthetized with pentasorbital sodium and transduced intranasally with 2.5×10⁸ FFU of Ad5-ACE2 in 50 μL DMEM. Five days post transduction, 1 hr before infection, these mice received either a dose of 15 mg/kg GA intraperitoneal injection (i.p.) in a volume of 150 μL, or an equivalent volume of Normal Saline (NS, 0.9% sodium chloride) administered to control mice. These mice were then infected intranasally with SARS-CoV-2 (1×10⁵ PFU) in a total volume of 50 μL DMEM. Infected mice continued to receive either GA or Normal Saline (NS) i.p. treatment for three days. All mice were weighted every day and euthanized at 4dpi. The mice lungs were collected and weighed; and lung homogenates were prepared in NS (0.1 g tissue with 0.5 mL NS) by crushing for 10 min and then centrifuging at 3000 rpm for 10 min at 4° C. The 100 μL supernatant of the lung homogenates were collected to extract viral RNA and qRT-PCR were used to assess the SARS-CoV-2 RNA copies in the infected lungs. All mice were euthanized after 4 day's treatments, the body weight loss, SARS-CoV-2 RNA copies in the lungs, lung pathological changes, key inflammatory cytokine levels (IL-6, IL-1β, TNF-α) in the lungs were studied.

### Pathological Examination of SARS-CoV-2 Infected and GA Treated Mice.

Experimental mice were anesthetized and the lungs were collected and were fixed in 4% (v/v) paraformaldehyde solution for 48 hours, and paraffin sections (3-4 μm) were prepared. The paraffin sections were stained with Hematoxylin and Eosin (H&E) to identify histopathological changes in the lungs. The histopathology images of the lung tissues were observed by light microscopy. All experiments with SARS-CoV-2 were conducted in the Biosafety Level 3 (BSL3) Laboratories of National Institute for Viral Disease Control & Prevention, Chinese Center for Disease Control and Prevention.

### Bio-distribution, Side Effects, and Pharmacokinetics Study of GA Gold Compounds in Mice or Rats.

BALB/c female mice and SD rats for experiments were conducted in compliance with regulations of the National Act on the use of experimental animals (China) and were approved by the Institutional Animal Care and Ethic Committee at the Chinese Academy of Sciences (approved No. SYXK (jing) 2014-0023). The experiment mice were intraperitoneally injected with GA at a dose of 15 mg/kg for 4 times (once a day for 4 days). Six hours after the last GA injection, the mice were anesthetized, and half of the organ tissues were analyzed by ICP-MASS to determinate the distributions of Au atoms in blood, brain, heart, lung, liver, spleen, and kidney tissues. The other half of the organ tissues were fixed in 4% (v/v) paraformaldehyde solution for 48 hours, and paraffin sections (3-4 μm) were prepared. The paraffin sections were stained with Hematoxylin and Eosin (H&E) to identify histopathological changes. The Au content in the various organ tissues were measured with ICP-MS (Thermo-X7). For male and female SD rats, after intraperitoneal injection or intravenous injection of 5 mg Au/kg.bw, respectively, blood were collected from jugular vein at different time points. The blood level of Au was analyzed with ICP-MS (Thermo-X7). PK parameters were determined using a noncompartmental analysis with PKSolver.

In reference to FIG. 13, Panel (a) shows the time scheme of administering GA or NS in relation to the infection of mice with SARS-CoV-2 virus intranasally. The COVID-19 mice model was successfully generated. Panels (b) to (d) show that comparative results from the SARS-COV-2 infected mice treated by NS and GA. There was more body weight loss (Panel (b)), higher SARS-CoV-2 RNA copies in lung (Panel (c)) and significant more severe bronchopneumonia and interstitial pneumonia and infiltration of lymphocytes within alveolar were observed (Panel (d)). The pathological scores of mice lung tissue were assessed by grading the injury from 0 to 4 in accordance with the INHAND scoring standard, the average pathological score of virus infected mice treated by NS is about ˜3. However, the body weight loss of GA treated mice was less compared with that of NS treated COVID-19 mice. The number of viral RNA copies in the lungs of GA treated mice were about ˜4×log10⁴, significantly lower than those in the lungs of NS treated infected mice which was ˜5×log10⁵.

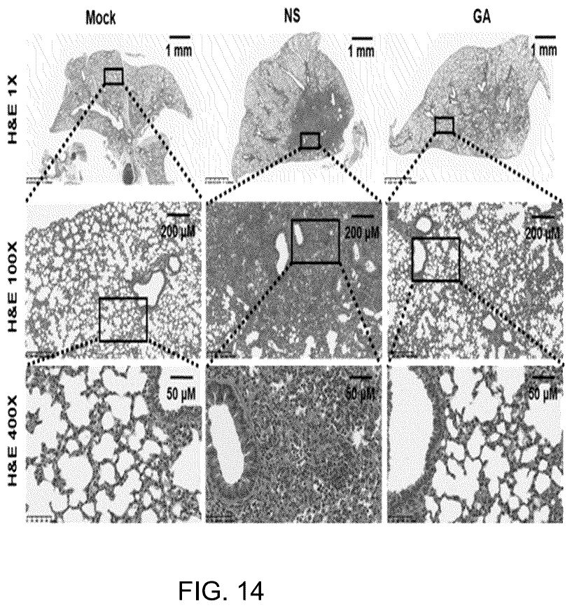

In reference to FIG. 14, histopathological analyses of the lung tissues of the GA treated COVID-19 mice in comparison with NS treated COVID-19 mice were shown. The SARS-CoV-2 infected mice treated with NS showed severe lung inflammation. The alveolar septum, bronchus, bronchioles and perivascular interstitium were significantly widened, along with more lymphocytes and a small number of neutrophils infiltration. Also, a small number of lymphocytes and exfoliated epithelial cells were found in the lumen of local bronchioles of NS treated mice. However, GA treated mice abrogated the characteristic signs of lung inflammation in SARS-CoV-2 infected mice. Local alveolar septum, bronchi, bronchiole and perivascular interstitial widening significantly decreased. Although there were still some lymphocytic infiltrations, the mucosal epithelium of bronchus and bronchioles were intact, and there were no foreign cells in the lumen, which showed comparable level to those lung tissue sections of the mock mice that were not infected by the SARS-CoV-2. The mean pathological score obtained from histopathological lung observation further demonstrated that GA gold cluster significantly (p<0.001) reduced pathological scores (˜1.8) compared with those of SARS-CoV-2 infected mice treated with NS (˜3.0).

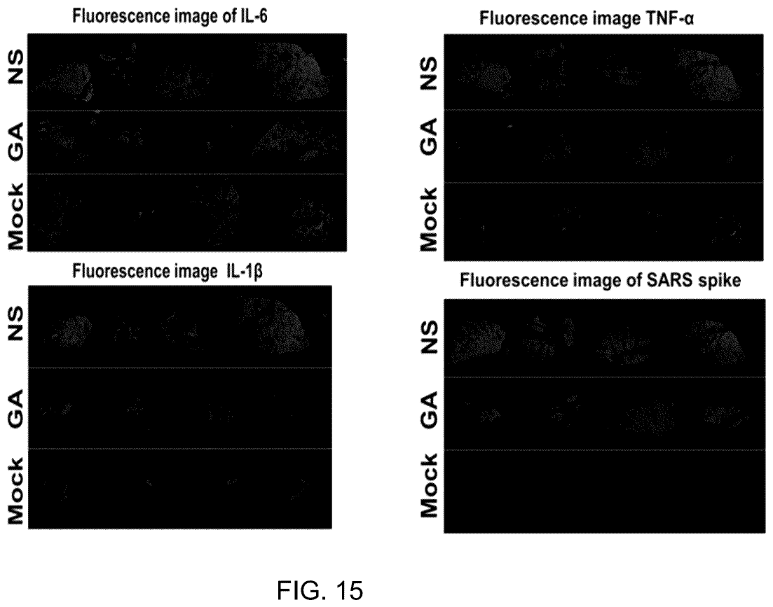

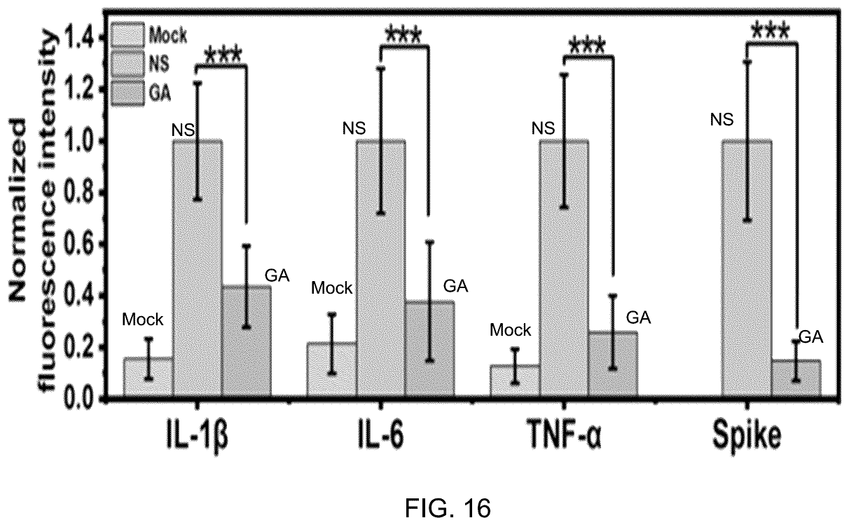

In reference to FIG. 15, the levels of the inflammatory cytokines in the lungs of COVID-19 mice were shown by immuno-fluorescence imaging. The levels of IL-6, IL-1β, TNF-α in the lungs of GA treated COVID-19 mice were visibly lower than those of NS treated COVID-19 mice. The GA treatment of COVID-19 mice thus significantly protected the lungs from injury by both inhibiting virus replication and suppressing the inflammatory cytokine expression in SARS-CoV-2 infected mice. FIG. 16 quantitatively shows the levels of IL-6, IL-1β, TNF-α in the lungs of GA and NS treated COVID-19 mice. The levels of cytokines were significantly lower in GA treated COVID-19 mice.

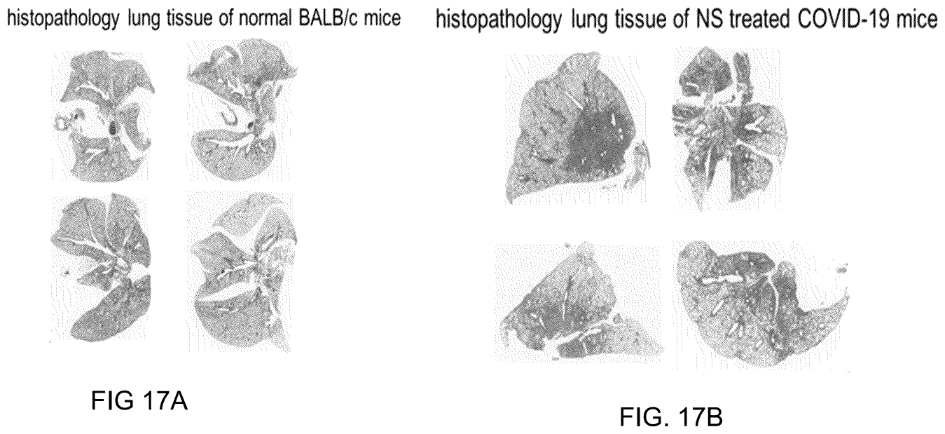

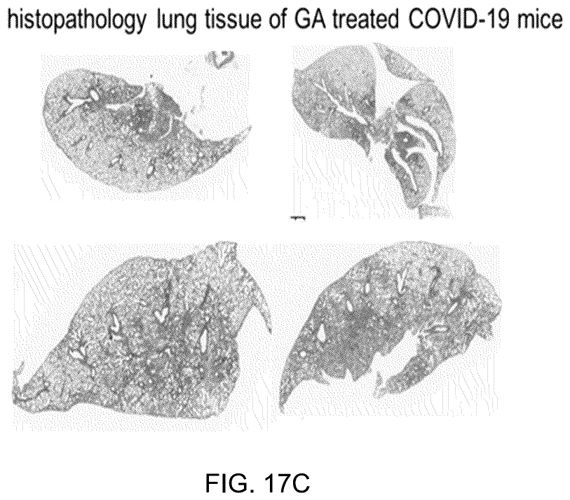

In reference to FIG. 17A, 17B and 17C, the pathological images of the lungs from normal mice, the NS treated COVID-19 mice and the GA treated COVID-19 mice are shown. The SARS-CoV-2 infected mice treated with NS showed severe lung inflammation (FIG. 17B). The alveolar septum, bronchus, bronchioles and perivascular interstitium showed lymphocytes and neutrophils infiltration. Also, a small number of lymphocytes and exfoliated epithelial cells were found in the lumen of local bronchioles of NS treated mice. It appears that treatment with GA for four days abrogated the characteristic signs of lung inflammation in SARS-CoV-2 infected mice (FIG. 17C). Local alveolar septum, bronchi, bronchiole and perivascular interstitial widenings w significantly decreased. There was much less lymphocytic infiltration, and the mucosal epithelium of bronchus and bronchioles were intact, no foreign body were found in the lumen, the lumen images were in comparable shape to those lung tissue sections of those normal mice that were not infected by the SARS-CoV-2 (FIG. 17A).

### The Bio-distribution, Tissue Pathologic, and Pharmacokinetic Studies of GA in Mice/Rat

To check the tissue distribution of Au ingredient and see if Au ingredient induced tissue side effects, six normal BLAC/C mice in the treatment group were intraperitoneally injection of 15 mg/kg.bw GA 4 times for 1 time/day, and the mice in the control group were injected with NS in the same way. During this study, no side effects in the GA treated mice were observed on aspects of movement, out looking, sleeping, and eating behaviors.

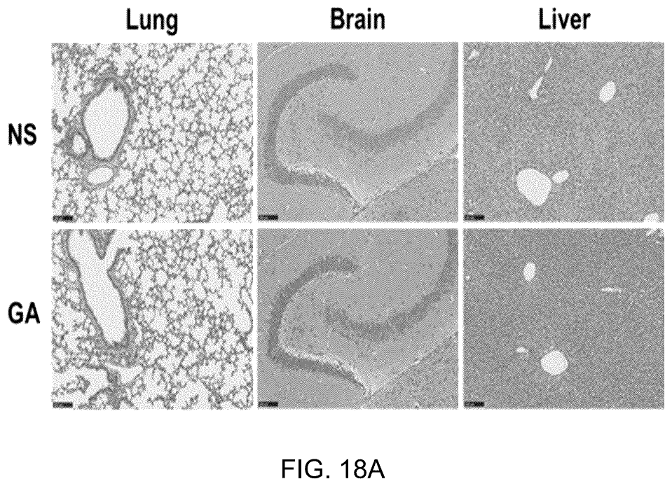

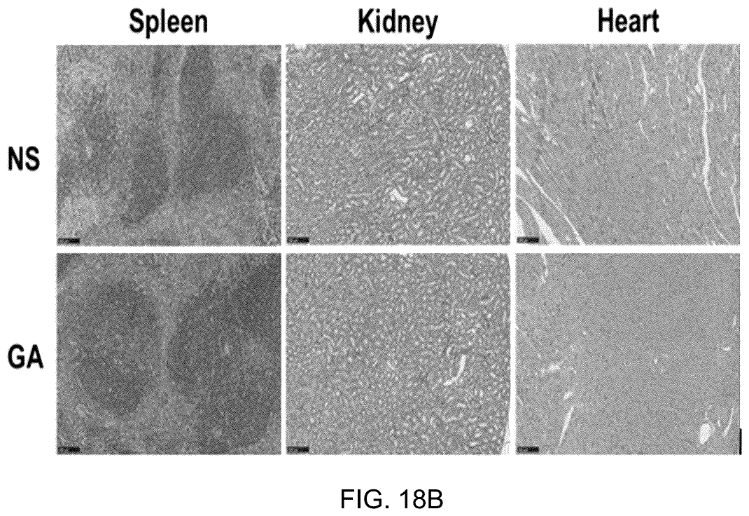

In reference to FIG. 18A and 18B, the pathological images of mouse brains, hearts, livers, lungs, spleens, and kidneys were shown after treating normal mice with GA for four days. Mouse tissue sections dyed by Hematoxylin-eosin (HE). No statistically significant pathological changes were found in these tissues from the GA treated normal mice compared with those of NS treated normal mice, which suggests that 15 mg/kg.bw GA treatment to the mice should be safe for the mice in this study.

The Au ingredient distribution in mice organs were analyzed by ICPMASS and the results are shown in Table III. In the lung, the Au element concentration is ˜51.07 μg/g, which may account for the GA related inhibition of virus replication and the suppressing inflammatory cytokine expression. The gold distribution in hearts, livers, kidneys, brains, and spleens can be beneficial for COVID-19 treatment as they may potentially inhibit SARS-CoV-2 replication and suppress the inflammation cytokine level in those organs. As shown in Table III, the Au ingredient mainly concentrated on spleen, heart, and kidney. The high level of Au in kidney implied the Au ingredient may quickly excrete via urine, this is consistent with the pharmacokinetics data of GA in the rat model shown in Table IV.

The pharmacokinetics parameters of GA gold cluster via intraperitoneal injection of rats were Table IV. After rats were intraperitoneally injected with 5 mg/kg.bw of GA for one time, Au concentrations in the plasma were tested at different time points and kinetic characteristics of gold cluster in rats were analyzed. According to the calculated parameters, the values of Tₘₐₓ for GA in male or female rats were 2 hours and the values of Cₘₐₓ for GA in male or female rats were 29.99 μg/mL or 31.750 μg/mL, respectively. The values of t ½ z for GA in male or female rats were 21.626 hr or 11.068 hr, respectively. Combine the data analysis of intravenous injection of GA at 5 mg/kg.bw, the F values of bioavailability for GA in male or female rats were 92.06% or 96.41%, respectively. These data confirm that GA has a favorable in vivo bioavailability in terms of pharmacological values.

TABLE III
Au distribution in mice tissues

| | Concentration |
|---|---|
| Tissue | (μg/g) |
| plasma | 15.01 ± 0.30 |
| brain | 2.52 ± 0.90 |
| heart | 10.35 ± 3.01 |
| lung | 51.70 ± 9.90 |
| spleen | 294.72 ± 12.35 |
| liver | 312.65 ± 7.42 |
| Spleen | 623.64 ± 22.66 |

TABLE IV
Pharmacokinetics of intraperitoneal injection GA in SD rats

| | | value | |
|---|---|---|---|
| parameter | unit | Female | Male |
| AUC₍₀₋ₜ₎ | mg/L*h | 533.680 | 509.615 |
| AUC_{(0-∞)} | mg/L*h | 599.607 | 785.906 |
| AμMC₍₀₋ₜ₎ | | 6371.497 | 7609.285 |
| AμMC_{(0-∞)} | | 9797.793 | 26177.773 |
| MRT₍₀₋ₜ₎ | h | 11.939 | 14.931 |
| MRT_{(0-∞)} | h | 16.340 | 33.309 |
| VRT₍₀₋ₜ₎ | h\hat{ }2 | 83.183 | 123.090 |
| VRT_{(0-∞)} | h\hat{ }2 | 258.916 | 1045.121 |
| t_{1/2z} | h | 11.068 | 21.626 |
| Tₘₐₓ | h | 2 | 2 |
| CL_{z}/F | L/h/kg | 0.008 | 0.006 |
| V_{z}/F | L/kg | 0.133 | 0.199 |
| Cₘₐₓ | mg/L | 31.750 | 29.990 |
| F | % | 96.407 | 92.060 |

As will be recognized by those skilled in the art, the innovative concepts described in the present application can be modified and varied over a tremendous range of applications, and accordingly the scope of patented subject matter is not limited by any of the specific exemplary teachings given. It is intended to embrace all such alternatives, modifications and variations that fall within the spirit and broad scope of the appended claims.

Additional general background, which helps to show variations and implementations, may be found in the following publications, all of which are hereby incorporated by reference herein for all purposes:

None of the description in the present application should be read as implying that any particular element, step, or function is an essential element which must be included in the claim scope: THE SCOPE OF PATENTED SUBJECT MATTER IS DEFINED ONLY BY THE ALLOWED CLAIMS. Moreover, none of these claims are intended to invoke paragraph six of 35 USC section 112 unless the exact words “means for” are followed by a participle.

The claims as filed are intended to be as comprehensive as possible. and NO subject matter is intentionally relinquished, dedicated, or abandoned.

What is claimed is:

1. A method for treatment of a coronavirus infection in an animal, comprising the steps of:
providing a gold compound as an active gradient that is effective in treating Rheumatoid Arthritis in said animal wherein a gold atom forms a gold-S bond in the coronavirus' protease active pocket and the gold compound suppresses inflammations in the animal's body; and
administering said gold compound to the animal having coronavirus infection via oral intake.

2. The method of claim 1, wherein the gold compound is AURANOFIN having molecule formula C₂₀H₃₄AuO₉PS⁰.

3. The method of claim 1, wherein the gold compound is Aurothioglucose having molecule formula C₆H₁₁AuO₅S.

4. The method of claim 1, wherein the gold compound is gold cluster complex wherein multiple gold atoms and multiple peptides or proteins or polymers form a complex molecule, and the gold atoms emit fluorescent light under excitation.

5. The method of claim 4, wherein the gold compound is a gold cluster complex having a molecular formula Au₂₉(SG)₂₇ wherein SG represents a glutathione peptide (SEQ. NO. 1) that Au represents a gold atom.

6. The method of claim 5 wherein the coronavirus infection is COVID-19 and the coronavirus is SARS-CoV-2.

7. The method of claim 1, wherein said step of administering is through intraperitoneal-injection or intramuscular-injection or intravenous-injection method.

8. The method of claim 1, wherein said step of administering is through nasal inhaling method.

9. The method of claim 1, wherein said step of providing a gold compound comprises the step of reacting gold (I) or gold (III) salt with glutathione peptide (SEQ. NO. 1) solutions.

10. The method of claim 1, wherein the sufficient amount is in the range of 1 mg/kg.bw to 20 mg/kg.bw of the animal.

11. A method for treatment of a coronavirus infection in an animal, the coronavirus encoding a conserved papain-like main protease critical for its replication, comprising the steps of:
providing a gold cluster compound having a molecular formula Au₂₉(SG)27 wherein SG represents glutathione peptide (SEQ. NO. 1) and Au represents gold atom, wherein said gold cluster compound functions as an active gradient that both forms gold-S bond in the active pocket of the main protease and suppresses cytokine expressions in the animal's body.
administering said gold compound to the animal having coronavirus infection via oral intake.

12. The method of claim 14, wherein the coronavirus infection is COVID-19 and the coronavirus is SARS-CoV-2.

13. The method of claim 14, wherein the gold cluster compound is administered to said animal in the range of 1 mg/kg.bw to 20 mg/kg.bw of the animal.

14. The method of claim 14, wherein the gold cluster compound is administered to said animal for at least 4 days.

15. The method of claim 14, wherein the gold cluster compound is administered to said animal through intraperitoneal-injection or intramuscular-injection or intravenous-injection method.

16. The method of claim 14, wherein the gold cluster compound is administered to said animal through nasal inhaling method.

17. The method of claim 14, wherein the gold cluster compound is administered to said animal through oral-intake method.

18. A therapeutic composition for treatment of a coronavirus infection in an animal, comprising:
about 150 μl liquid solution of about 15 mg of the gold cluster complex having a molecular formula Au₂₉(SG)₂₇ wherein SG represents a glutathione peptide (SEQ. NO. 1) wherein the liquid solution contains 0.9% (w/w) sodium chloride.

19. A method for making a therapeutic composition for treatment of a coronavirus infection in an animal comprising the steps of:
reacting glutathione solution with chloroauric acid solution at ambient temperature;
dialyzing said reaction solution;
filtering said dialyzed reaction solution through a molecular weight cut resin spin column;
collecting the filtered portion of molecule weight corresponding to that of Au₂₉(SG)₂₇; and
determining Au₂₉(SG)₂₇ molecule structure using electrospray ionization mass spectrometry.
